(19) United States
(12) Patent Application Publication
(10) Pub. No.: US 2008/0213356 A1
(43) Pub. Date: Sep. 4, 2008

(54) Pharmaceutical Composition Containing Hmg-Coa Reductase Inhibitor And Method For The Preparation Thereof

(75) Inventors: Evangelos Karavas, Athens (GR); Eftimios Koutris, Athens (GR); Dimitrios Bikiaris, Thessaloniki (GR)

Correspondence Address: AKC PATENTS, 215 GROVE ST., NEWTON, MA, 02466, US

(73) Assignee: PHARMATHEN S.A., Pallini-Attikis (GR)

(21) Appl. No.: 12/063,222

(22) PCT Filed: Sep. 13, 2006
(86) PCT No.: PCT/GR2006/000046

§ 371 Date: Feb. 7, 2008

(30) Foreign Application Priority Data
Sep. 14, 2005 (GR) 20050100466

(51) Int. Cl. A61K 9/48; A61K 31/404; A61K 31/40; A61K 9/20

(52) U.S. Cl. 424/451; 514/419; 514/423; 424/464

(57) ABSTRACT

The present invention relates to the formulation of solid dosage forms comprising a therapeutically effective amount of an HMG-CoA reductase inhibitor, and especially Fluvastatin or Atorvastatin or salts thereof, in combination with inorganic silica polymer such as Dimethicone, and a process for the preparation thereof by direct compression.

### TECHNICAL FIELD OF THE INVENTION

The present invention relates to improved dosage forms such as tablets and capsules and in particular to a formulation for oral or sub-lingual administration comprising a therapeutically effective quantity of a HMG-CoA reductase inhibitor, and more particularly Fluvastatin, Atorvastatin or salts thereof, in combination with inorganic silica polymer such as Dimethicone and a method for the preparation thereof.

### BACKGROUND OF THE INVENTION

HMG-CoA reductase inhibitors, commonly known as “statins”, act through the inhibition of 3-hydroxy-3-methylglutaryl-coenzyme A (HMG-CoA) reductase. This enzyme catalyzes the conversion of HMG-CoA to mevalonate, an early and rate-limiting step in cholesterol biosynthesis. Statins are useful in the treatment of hyperlipoproteinemia and atherosclerosis but are extremely susceptible to degradation at pH below 8. Statins at pH below 8 and particularly in acidic conditions, undergo elimination or isomerization or oxidation reactions to form conjugated unsaturated aromatic compounds, as well as the threo isomer, the corresponding lactones and other degradation products. Statins are particularly sensitive to an acidic environment (a low pH environment), in which hydroxyl acids are degraded into lactone. The tendency of HMG-CoA reductase inhibitors to degrade may be accelerated by possible interactions with other active ingredients or excipients present in the composition.

Fluvastatin sodium, is the [R*,S*-(E)]-(±)-7-[3-(4-fluorophenyl)-1-(1-methylethyl)-1H-indol-2-yl]-3,5-dihydroxy-6-heptenoic acid, monosodium salt and Atorvastatin calcium, is the [R—(R*,R*)]-2-(4-fluorophenyl)-b,d-dihydroxy-5-(1-methylethyl)-3-phenyl-4 [(phenylamino) carbonyl]-1H-pyrrole-1-heptanoic acid, calcium salt (2:1) trihydrate. Fluvastatin and Atorvastatin are two statins particularly useful in therapeutics but prone to degradation reactions. The degradation of the active ingredient results in reduced effectiveness and treatment failure.

Furthermore, the stability of pharmaceutical compositions containing a HMG-CoA reductase inhibitor and in particular, Fluvastatin or Atorvastatin or salts thereof can also be influenced by the selection of the excipients.

Moreover, the poor flow properties of certain Statins may also generate difficulties when it has to be formulated in dosage forms suitable for oral or sub-lingual administration, such as tablets, capsules, caplets, sachets or other solid dosage forms, thus limiting the choices of the excipients that can really be used.

The bioavailability and the release rate of the pharmaceutical dosage can also be enhanced by the selection of the excipients.

Various methods are already known for the industrial preparation of oral dosage forms comprising a HMG-CoA reductase inhibitor e.g. Fluvstatin or Atorvastatin or salts thereof, as an active ingredient due to its useful therapeutical properties. However, the prior art has encountered substantial difficulties in the production of the oral solid formulations of a desirable stability due to the degradation of said active ingredient.

EP 0 547 000 discloses a pharmaceutical composition which comprises a statin and an alkaline stabilizing medium capable of imparting a pH of at least 8 to an aqueous solution or dispersion of the composition.

EP 1 148 872 discloses a stable solid pharmaceutical formulation comprising a statin and a buffering agent, such as a carbonate buffer or phosphate buffer, capable of adjusting the pH of the total formulation in the range from 7 to 11.

Furthermore, in EP 1 292 293 is disclosed a composition comprising a homogenous mixture of a statin with a buffering or basifying substance obtained by co-crystallization and/or co-precipitation of the statin and the buffering or basifying substance.

Although each of the above patents represents an attempt to overcome the instability problems associated with pharmaceuticals compositions comprising a HMG-CoA reductase inhibitor, there still exists a need for improving the stability of such pharmaceutical compositions.

### SUMMARY OF THE INVENTION

It is, therefore, an object of the present invention to provide an improved solid dosage formulation for oral or sub-lingual administration containing a HMG-CoA reductase inhibitor, and in particular Fluvastatin or Atorvastatin or salts thereof as an active ingredient, which overcomes the deficiencies of the prior art and avoids the degradation of the active ingredient.

Another aspect of the present invention is to provide a solid dosage formulation for oral or sub-lingual administration containing a HMG-CoA reductase inhibitor, and in particular Fluvastatin or Atorvastatin or salts thereof as an active ingredient, which is bioavailable and effective with sufficient self-life and good pharmacotechnical properties.

Moreover, another aspect of the present invention is to provide a solid dosage formulation for oral or sub-lingual administration containing a HMG-CoA reductase inhibitor, and in particular Fluvastatin or Atorvastatin or salts thereof as an active ingredient, which can be prepared in dosage forms of different strength by proportionally adjusting the quantities of the excipients and the active ingredient, thereby providing a pharmacotechnical linearity, without affecting the dissolution profile and bioavailability of the active ingredient.

A further aspect of the present invention is to provide a method for the preparation of a stable solid dosage formulation for oral or sub-lingual administration containing a HMG-CoA reductase inhibitor, and in particular Fluvastatin or Atorvastatin or salts thereof as an active ingredient, thereby stabilizing said active ingredient and improving the flow properties and the pharmacotechnical characteristics of the composition.

Still, another aspect of the present invention is to provide a method of improving the flow properties of a solid dosage formulation for oral or sub-lingual administration containing a HMG-CoA reductase inhibitor, and in particular Fluvastatin or Atorvastatin or salts thereof as an active ingredient.

In accordance with the above objects of the present invention, a pharmaceutical composition for oral or sub-lingual administration is provided comprising a HMG-CoA reductase inhibitor or a pharmaceutical acceptable salt thereof as an active ingredient, and an effective amount of inorganic silica polymer such as Dimethicone as a stabilizer to inhibit isomerization and/or elimination and/or oxidation.

According to another embodiment of the present invention, a process for the preparation of solid dosage forms for oral or sub-lingual administration such as tablets, capsules and sachets, containing a HMG-CoA reductase inhibitor or a pharmaceutical acceptable salt thereof as an active ingredient is provided, which comprises:
Forming a homogenous mixture by mixing the total quantity of said active ingredient with a total quantity of inorganic silica polymer such as Dimethicone as a stabilizer to inhibit isomerization and/or elimination and/or oxidation;
Sieving the above mixture through a sieve;
Adding to the sieved mixture the total quantities of at least one optionally excipient such as a binder, a diluent, a disintegrant, a lubricant and/or a glidant and mixing until uniform, and
Formulating the resulting mixture in a solid dosage form either by compressing it into a desired tablet form or by filling capsules or sachets.

Alternative processes for the preparation of the pharmaceutical composition according to the present invention are also defined in independent claims 13 and 14.

Further preferred embodiments of the present invention are defined in dependent claims 2 to 11 and 15 to 17.

Other objects and advantages of the present invention will become apparent to those skilled in the art in view of the following detailed description.

### BRIEF DESCRIPTION OF THE DRAWINGS

FIG. 1 shows a X-RD spectrum of an amorphous Fluvastatin and a mixture of Fluvastatin and Dimethicone according to the present invention directly after the preparation and after 6 months of storage in the conditions of an accelerated ageing (40° C., 75% RH).

FIG. 2 shows a FT-IR spectrum of Dimethicone, Fluvastatin and a mixture of both in 50%/50% by weight according to the present invention.

### DETAILED DESCRIPTION OF THE INVENTION

For the purposes of the present invention, a pharmaceutical composition comprising an active ingredient (HMG-CoA reductase inhibitor e.g. Fluvastatin or Atorvastatin or salts thereof) is considered to be “stable” if said ingredient degradates less or more slowly than it does on its own and/or in known pharmaceutical compositions.

An excipient is considered to be “incompatible” with an active ingredient (HMG-CoA reductase inhibitor e.g. Fluvastatin or Atorvastatin or salts thereof) if it promotes the degradation of said active ingredient, that is to say, if said active ingredient (HMG-CoA reductase inhibitor e.g. Fluvastatin or Atorvastatin or salts thereof) degrades more or faster in the presence of said excipient when compared with the degradation of said active ingredient (HMG-CoA reductase inhibitor e.g. Fluvastatin or Atorvastatin or salts thereof) on its own. The terms “incompatibility”, “compatible” and “compatibility” are defined accordingly.

The active ingredient (HMG-CoA reductase inhibitor e.g. Fluvastatin or Atorvastatin or salts thereof) contained in a dosage form is “bioavailable”, if when administered in a dosage form is released from the dosage form, absorbed and reaches, at least the same, concentration levels in plasma as any of the marketed products containing the same quantity of the same active ingredient and intended for the same use.

Although the pharmaceutical composition may be in various forms, the preferred solid forms are tablets, capsules and caplets.

The improved solid pharmaceutical composition of the present invention is characterized by physicochemical properties suitable for the tablet formulation by direct compression, the adequate release rate of the active ingredient (HMG-CoA reductase inhibitor e.g. Fluvastatin or Atorvastatin or salts thereof) and the storage stability, by employing excipients practically devoiding the tendency to interact with the active ingredient, and possessing good compressibility properties.

As already mentioned certain HMG-CoA reductase inhibitors are susceptible to degradation/oxidation and their tendency gets stronger when they are formulated and mixed with excipients or other active substances.

Moreover, certain HMG-CoA reductase inhibitors such as Fluvastatin or Atorvastatin or salts thereof, have a relative low bulk density, poor flow properties and stick to metal surfaces during tableting. It is, therefore, necessary to employ at least a lubricant in the tablet formulation of said compositions, in order to reduce the friction during tablet compression. The lubricant deforms easily when sheared between two surfaces and, hence, when interposed between the tablet and the die wall, provides a readily deformable film that eliminates the friction between the compressed tablet and the die, so that the tablet can be removed from the die without damage.

In addition, the inclusion of a glidant is also necessary, in order to improve the flow properties for sufficient and uniform die filling. This is achieved as the glidant lodges in the irregularities of the granule surface, forming a more rounded structure and thus reducing interparticulate friction.

It must be stressed that the functions of a glidant and a lubricant in formulation processes are totally different. A few materials e.g. talc, can act, at the same time, as glidant and lubricant, but usually two different excipients are required.

One of the main disadvantages of the HMG-CoA reductase inhibitors is the fact that, they are very labile to acidic pH environment, and consequently many limitations concerning the choice of excipients are raised.

Moreover, the lubricant and the glidant should be very carefully selected because some of them are very hydrophobic and affect negatively the disintegration and dissolution while it has been shown to cause bioavailability problems. The manufacturing process should also be very carefully determined because relatively high concentrations of lubricant and/or glidant reduce crashing strength and increase disintegration time especially when associated with prolonged mixing times.

Furthermore, it is already known either to include alternative excipients as lubricants with or without stabilizing agents, or to use more complicated formulations and/or manufacturing processes.

In addition, the use of Hydrogenated Vegetable Oil can cause processing problems of sticking to the punches during long tableting runs, Sodium Stearyl Fumarate is much more expensive than other commonly used lubricants and causes similar tablet strength reduction and prolongation of disintegration time. Moreover, Glyceryl Dibehenate has the least anti-adherent effect and it is required at higher levels than, for example, Magnesium Stearate or Sodium Stearyl Fumarate, for effective lubrication, and besides, it has a not negligible retardant effect on dissolution rate.

It has been surprisingly found that the object of the present invention is achieved by employing a low and/or medium density inorganic silica polymer such as Dimethicone as a stabilizer, while its nominal viscosity may range from 50 cSt to 1000 cSt.

In fact, when Dimethicone is incorporated in a pharmaceutical composition according to the present invention, it is not necessary to employ an additional buffering or alkaline agent in order to avoid the degradation of statins.

Dimethicone is an amorphous liquid macromolecular silicon oil, and more specifically a linear polydimethysiloxane (polymer) with a degree of polymerisation in the range of n=20 to 400 and a nominal kinematic viscosity in the range of 20 to 1300 mm²/s (from 20 cSt to 1300 cSt). Dimethicone is commercially available under different grades of density and various viscosities. Moreover, dimethicone is a hydrophobic oily substance, practically insoluble in water, resistant to heat and stable during storage.

When dimethicone is incorporated in a pharmaceutical composition according to the present invention it is adsorbed by the crystal or amorphous particles of the active ingredient resulting in a one-phase system. In this system Dimethicone and the active ingredient are in contact in a molecular basis. Said one-phase system protects the active ingredient from oxidation and/or elimination and/or isomerization. Thus, Dimethicone serves as a protective barrier, isolating the active ingredient against humidity and/or air oxygen and/or a low pH environment. Moreover, the crystals of the active ingredient become soften and more regular in shape leading in improved compressibility and better flowability. The protection of the active ingredient may be partially attributed to the hydrophobic character of Dimethicone. This, however, unexpectedly does not affect the dissolution rate of the active substance as it is used in such a proportion that its emulsifying properties prevail and therefore excellent bioavailability is achieved.

One of the main objects of the present invention was to prepare a product with an acceptable stability. For this reason samples of Fluvastatin alone, Fluvastatin with Dimethicone, and Fluvastatin with Dimethicone and Colloidal Silicon Dioxide were exposed to accelerated stability tests at a temperature of 65° C. and 40° C. and relative humidity of 75% for 20 days.

The results show that fluvastatin degrades less and is more stable when dimethicone is incorporated in fluvastatin. The stabilization effect is even more enhanced by the additional incorporation of Colloidal Silicon Dioxide

The specific tests and results are described in the stability table (TABLE 1 and 2).

TABLE 1
STABILITY AT 65° C. TEMPERATURE AND 75% RELATIVE HUMIDITY

| | | Fluvastatin- | Fluvastatin-Dimethicone- |
|---|---|---|---|
| 65° C., 20 days | Fluvastatin | Dimethicone | Colloidal Silicon Dioxide |
| Fluvastatin anti-isomer | 0.37% | 0.29% | 0.23% |
| 3-hydroxy-5- | 0.26% | 0.18% | 0.15% |
| keto fluvastatin | | | |
| Fluvastatin t- | ND | ND | ND |
| butyl ester | | | |
| Fluvastatin | ND | ND | ND |
| Hydroxy-diene | | | |
| Any individual | RRT 1.25: 0.05% | RRT 1.25: 0.04% | RRT 1.25: 0.04% |
| unknown | RRT 1.54: 0.15% | RRT 1.54: 0.12% | RRT 1.54: 0.11% |
| | RRT 1.87: 0.13% | RRT 1.87: 0.11% | RRT 1.87: 0.11% |
| Total | 0.96% | 0.74% | 0.64% |

TABLE 2
STABILITY AT 40° C. TEMPERATURE AND 75% RELATIVE HUMIDITY

| 40° C./75% RH, | | Fluvastatin- | Fluvastatin-Dimethicone- |
|---|---|---|---|
| 20 days | Fluvastatin | Dimethicone | Colloidal Silicon Dioxide |
| Fluvastatin anti-isomer | 0.76% | 0.62% | 0.57% |
| 3-hydroxy-5- | 0.30% | 0.11% | 0.09% |
| keto fluvastatin | | | |
| Fluvastatin t- | ND | ND | ND |
| butyl ester | | | |
| Fluvastatin | ND | ND | ND |
| Hydroxy-diene | | | |
| Any individual | ND | RRT 1.25: 0.03% | ND |
| unknown | RRT 1.54: 0.04% | RRT 1.54: 0.04% | RRT 1.54: 0.04% |
| | RRT 1.87: 0.05% | RRT 1.87: 0.04% | RRT 1.87: 0.03% |
| Total | 1.15% | 0.84% | 0.73% |

The results show a good stability when Dimethicone is incorporated in Fluvastatin and an even better stability when silicon dioxide is incorporated in a composition comprising the active ingredient (Fluvastatin) and Dimethicone. This may be attributed to the fact that silicon dioxide lodges in the surface irregularities of the particles forming a more rounded shape and thus facilitates the adsorption of Dimethicone.

It has been confirmed through several tests such as FT-IR spectra analysis, DSC thermal analysis, and X-RD analysis, that active ingredient and Dimethicone according to the formulation of the present invention, are chemically inert and, thus, there is no chemical interaction between the two substances and all characteristic absorption peaks and bands of the active ingredient are present in the mixture, as well.

Furthermore, the active ingredient remained chemically untouched, showing the same melting point whether it is analysed in the mixture or by itself. In addition, the X-RD analysis showed that the crystal of the active ingredient remained invariable after mixed with Dimethicone. Besides SEM analysis revealed that the shape of the crystals did not change.

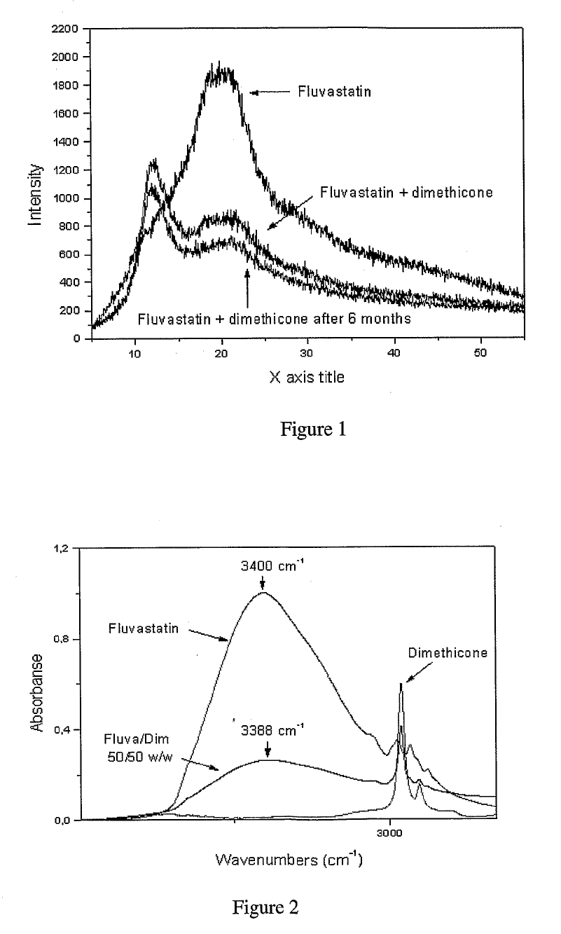

Moreover, as shown in FIG. 1, the mixture of an amorphous statin and Dimethicone does not promote the crystallization of said Statin, as it has been confirmed by X-RD analysis wherein all the recorded characteristic broad peaks were unchanged after 6 months storage in accelerated conditions (40° C. and 75% RH). This indicates that the crystal of the active ingredient remained invariable after mixed with Dimethicone. The crystal properties remain also unchanged after six months in the same conditions when the mixture is incorporated in a pharmaceutical composition with other excipients.

The stabilization could be the result of hydrogen bonding between the statin hydroxylic groups and Si—O— oxygen of dimethicone. Further, as shown in FIG. 2, FT-IR spectra analysis of Fluvastatin shows a strong broad absorbance band at 3400 cm⁻¹, wherein Dimethicone does not show any peak at this range. In the mixture of Fluvastatin-Dimethicone the same peak is appeared at 3388 cm⁻¹ probably due to interactions and hydrogen bond formation.

Specific tablet characteristics such as stability, resistance to crashing and friability are much more improved with the use of Dimethicone and even more with the use of Dimethicone and silicon dioxide.

A mixture of the active ingredient (HMG-CoA reductase inhibitor, especially Fluvastatin or Atorvastatin or salts thereof) with a suitable amount of inorganic silica polymer such as Dimethicone is formed, and subsequently admixed to complete homogeneity. After sieving the mixture, any optional additional excipient is then added. The composition is then mixed until uniform. The resulting composition may then be compressed.

The bulk density of the above composition is effectively reduced and the flow is impressively improved to such a degree that a glidant is not required any more, while, unexpectedly, the composition does not adhere to the tableting machine and uniform die filling is accomplished.

Moreover, any excipient may optionally be added to the above composition, provided that they are compatible with the active ingredient of the composition, in order to overcome problems associated with the poor flow properties and unfavorable pharmacotechnical characteristics of these substances, and in order to increase the stability of the drug and the self-life of the pharmaceutical product, and provide a product exhibiting excellent bioavailability.

The present invention can be applied in the formulation of tablets, capsules, caplets, sachets or other solid dosage forms for oral or sub-lingual administration of an active ingredient having stability problems especially related with oxygen and/or humidity of the atmosphere and/or acidic environment.

Another essential advantage of the present invention is that the solid dosage form according to the present invention ensures excellent bioavailability of the active ingredient. Furthermore, it is possible to prepare dosage forms of different strength using appropriate quantity of the same composition, thereby limiting the cost of production and minimizing the number, and consequently the cost, of clinical studies required for the approval of the product by the authorities.

The manufacturing process for preparation according to the present invention is simpler and inexpensive in comparison to the wet granulation process or any other conventional method.

Therefore, in a first embodiment, the present invention provides a pharmaceutical composition comprising from about 0.5% to 50% by weight of Fluvastatin or Atorvastatin or salts thereof and from about 0.1% to 25% by weight of Dimethicone. The weight ratio of Fluvastatin or Atorvastatin or salts thereof to Dimethicone is preferably 500:1 to 1:50 and more preferably 200:7.5 to 7.5:80.

Preferred pharmaceutical compositions according to the present invention comprise approximately 0.5% to 40%, more preferably 0.75% to 25% and most preferably 0.75% to 20% by weight of Fluvastatin or Atorvastatin or salts thereof.

More preferred pharmaceutical compositions according to the present invention comprise approximately 0.2% to 20%, more preferably 0.5% to 15% and most preferably 0.75% to 10% by weight of Dimethicone.

The preferred pharmaceutical compositions are in the form of solid dosage forms for oral or sub-lingual administration such as tablets, capsules, caplets, troches, pastilles, pills, lozenges and the like, in all shapes and sizes, coated or uncoated.

All percentages stated herein are weight percentages based on total composition weight, unless otherwise stated.

Another embodiment of the present invention is the use of the direct compression process for the preparation of solid dosage forms such as tablets containing HMG-CoA reductase inhibitor/Fluvastatin or Atorvastatin or salts thereof, which is one of the most economical methods.

The direct compression process of the present invention for the preparation of solid dosage forms for oral or sub-lingual administration such as tablets containing HMG-CoA reductase inhibitor such as Fluvastatin or Atorvastatin or salts thereof as an active ingredient comprises:
Forming a homogenous mixture by mixing the total quantity of the active ingredient with the total quantity of a suitable amount of inorganic silica polymer such as Dimethicone as a stabilizer to inhibit isomerization and/or elimination and/or oxidation and/or re-crystallization;
Sieving the above mixture through a sieve, and subsequently;
Adding to the sieved mixture the total quantities of at least one optional excipient such as a binder, a diluent, a filler, a disintegrant, a lubricant and/or a glidant and mixing until uniform, and
Formulating the resulting mixture in a solid dosage form either by compressing it into a desired tablet form or by filling capsules or sachets.

Alternatively, a blend of the total quantity of the active ingredient (HMG-CoA reductase inhibitor such as Fluvastatin or Atorvastatin or salts thereof) and the total batch quantity or a portion thereof of an optional diluent, and/or the total batch quantity or a portion thereof of an optional filler is formed and subsequently is admixed with the total batch quantity of an effective amount of Dimethicone to complete homogeneity. Then any other optional auxiliary excipient may be added.

The final mixture of the composition can be compressed into tablets or caplets, filled into capsules, or processed into another solid form.

Alternatively, a wet granulation process may also be used for the preparation of the pharmaceutical composition of the present invention. Said wet granulation process comprises:
forming a first blend of the total quantity of the active ingredient (HMG-CoA reductase inhibitor, such as Fluvastatin or Atorvastatin or salts thereof) with an effective amount of inorganic silica polymer, such as Dimethicone, as a stabilizer and colloidal silica dioxide and optionally the total batch quantity or a portion thereof of an optional diluent, and/or the total batch quantity or a portion thereof of an optional filler and wet granulating by the addition of a water-free granulating medium (i.e. absolute ethanol, acetone);
drying the wetted mass,
sieving the dried material to achieve the desired granule size
forming a second blend by mixing the total quantity of any other optional excipient such as a binder, a disintegrant, a lubricant, a colorant and/or a glidant until uniform and
adding and mixing the dried material with the second blend and
formulating the resulting mixture in a solid dosage form either by compressing it into a desired tablet form or by filling capsules or sachets.

The pharmaceutical compositions of the present invention are characterized by excellent pharmacotechnical properties, such as homogeneity, flowability and compressibility. Thanks to these properties, the solid dosage forms prepared by the above process exhibit excellent technical characteristics including disintegration time, dissolution rate, hardness, resistance to crashing, friability and stability, as better illustrated by the following measurements during the stage of the development of the products.

Namely, the pure pharmaceutical substance Fluvastatin showed very limited flowability and compressibility with a mean Carr's Index of 38-40%, whilst Atorvastatin showed a better mean Carr's Index of 24-25%. However, when Dimethicone was incorporated according to the present invention, a decrease of 40-50% of the Carr Index was observed, which indicates an improvement of the flow properties of Fluvastatin and Atorvastatin and therefore the use of a direct compression for the final formulation.

The pharmaceutical formulations according to the present invention have excellent pharmacotechnical properties indicating the suitability of the process and of the selected excipients as well.

One of the most critical pharmacotechnical tests, is the Dissolution test as it is strongly correlated with the bioavailability of the product. For the dissolution method a Paddle Apparatus was used 50 rpm, 37° C., time 30 min, while as a dissolution medium 900 ml of H₂O was used.

The most preferable compositions described below were investigated for their scalability, while a process validation was performed in order to prove the repeatability and accuracy of the manufacturing process and the proposed formulations. For the above tests 3 batches per strength were used.

The validation process showed that the compositions and the manufacturing process are suitable in order to provide a repeatable and high quality product.

Namely, the appearance was found to be acceptable in all cases. The disintegration time was less than 8 min, the dissolution was found to be over 90% in 30 minutes, whilst the Assay was between 95% and 105%.

No degradation products were observed during and after the procedure.

One of the main objects of the present invention was to prepare a product with acceptable stability. For this reason 3 batches of each strength (10 mg, 20 mg, 40 mg and 80 mg) were exposed to normal and accelerated stability studies according to the current ICH guidelines.

The following compositions were used per strength.

| | Strength | | | |
|---|---|---|---|---|
| | 10 mg | 20 mg | 40 mg | 80 mg |
| Ingredients | mg per tablet | | | |
| Atorvastatin | 10.85 | 21.69 | 43.38 | 86.76 |
| Dimethicone | 2.5 | 5.0 | 10.0 | 20.0 |
| MCC | 10.0 | 20.0 | 40.0 | 80.0 |
| Lactose monohydrate | 11.0 | 22.0 | 44.0 | 88.0 |
| Starch 1500 | 11.0 | 22.0 | 44.0 | 88.0 |
| Primojel | 4.0 | 8.0 | 16.0 | 32.0 |
| SLS | 0.5 | 1.0 | 2.0 | 4.0 |
| Aerosil | 0.5 | 1.0 | 2.0 | 4.0 |
| Mg stearate | 0.5 | 1.0 | 2.0 | 4.0 |

The tablets were packed in into containers impervious to water vapor e.g. PVC/PVDC and stored in appropriate stability chambers at a temperature of 25° C.±2° C. and relative humidity of 60%±5% for normal conditions and at a temperature of 40° C. and relative humidity of 75% for accelerated conditions. The tablets were tested in predetermined time intervals.

Strength

| | |
|---|---|
| 20 mg | 40 mg |

| | |
|---|---|
| Ingredients | mg per capsule |

| | | |
|---|---|---|
| Fluvastatin Sodium | 21.10 | 42.20 |
| Dimethicone | 5.00 | 10.00 |
| MCC | 18.00 | 36.00 |
| Lactose monohydrate | 22.45 | 44.90 |
| Starch 1500 | 22.45 | 44.90 |
| Primojel | 8.00 | 16.00 |
| SLS | 1.00 | 2.00 |
| Aerosil | 1.00 | 2.00 |
| Mg Stearate | 1.00 | 2.00 |

The frequency of the testing, the specific tests and results indicated for each batch are described in the stability table (TABLE 3).

TABLE 3
STABILITY AT 40° C. ± 2° C. TEMPERATURE AND 75 ± 5%
RELATIVE HUMIDITY
Fluvastatin capsules 40 mg

| | | Time in Months | | |
|---|---|---|---|---|
| Control | Specification | 0 | 3 | 6 |
| Appearance | Brown-orange | No change | No change | No change |
| | No1 capsule | | | |
| Assay (by HPLC) | 95-105% | 101.2% | 100.4% | 99.3% |
| Disintegration Time | Max 15 min in | 4′20″-5′45″ | 4′15″-5′20″ | 4′25″-5′40″ |
| | water at 37° C. | | | |
| Dissolution (Paddles, | Each tablet >85% of | 101.2% | 100.2% | 99.1% |
| 900 ml water, 50 rpm | the stated amount in | | | |
| | 30 min | | | |
| Related substances | | | | |
| Anti-isomer | NMT 0.80% | 0.26% | 0.27% | 0.27% |
| 3-hydroxy-5-keto- | NMT 0.50% | 0.13% | 0.21% | 0.34% |
| fluvastatin | | | | |
| Fluvastatin tert-butyl- | NMT 0.20% | 0.03% | 0.05% | 0.09% |
| ester | | | | |
| Hydroxy-diene | NMT 0.10% | ND | ND | ND |
| Fluvastatin | | | | |
| Single unknown | NMT 0.20% | ND | 0.03% | 0.05% |
| TOTAL | NMT 1.80% | 0.07% | 0.24% | 0.36% |

The results show a good stability of the product and compatibility between the drug substance and the excipients proposed by the present invention. The excellent results regarding the physicochemical characteristics, the excellent stability of the product as well as the simple and economic manufacturing process indicate the advantages of the present invention relative to the commonly used methods and excipients for the formulation of Fluvastatin and Atorvastatin.

The pharmaceutical compositions of the present invention may also contain one or more additional formulation ingredients selected from a wide variety of excipients. According to the desired properties of the composition, any number of ingredients may be selected, alone or in combination, based upon their known uses in preparation of solid dosage form compositions.

Such ingredients include, but are not limited to, diluents, binders, compression aids, disintegrants, glidants, lubricants, flavors, water scavengers, colorants, sweetener, coating agents and preservatives.

The optional excipients must be compatible with the HMG-CoA reductase inhibitor or the salt thereof so that it does not interfere with it in the composition.

Diluents may be, for example, calcium carbonate, calcium phosphate dibasic, calcium phosphate tribasic, calcium sulfate, microcrystalline cellulose, microcrystalline silicified cellulose, powdered cellulose, dextrates, dextrose, fructose, lactitol, lactose anhydrous, lactose monohydrate, lactose dihydrate, lactose trihydrate, mannitol sorbitol, starch, pregelatinized starch, sucrose, talc, xylitol, maltose maltodextrin, maltitol.

Binders may be, for example, acacia mucilage, alginic acid, carbomer, carboxymethylcellulose calcium, carboxymethylcellulose sodium, microcrystalline cellulose, powdered cellulose, ethyl cellulose, gelatin, liquid glucose, guar gum, hydroxyethyl cellulose, hydroxypropyl cellulose, hydroxypropylmethyl cellulose, maltodextrin, methylcellulose, polydextrose, polyethylene oxide, povidone, sodium alginate, starch paste, pregelatinized starch, sucrose.

Disintegrants may be, for example, alginic acid, carbon dioxide, carboxymethylcellulose calcium, carboxymethylcellulose sodium, microcrystalline cellulose, powdered cellulose, croscarmelose sodium, crospovidone, sodium docusate, guar gum, hydroxypropyl cellulose, methylcellulose, polacrilin potassium, poloxamer, povidone, sodium alginate, sodium glycine carbonate, sodium laulyl sulfate, sodium starch glycolate, starch, pregelatinized starch.

Glidants may be, for example, calcium silicate, powdered cellulose, starch, talc, colloidal silicon dioxide.

Lubricants e.g. polyethylene glycol 4000, polyethylene glycol 6000, sodium lauryl sulfate, starch, talc.

Still another embodiment of the present invention is the use of inorganic silica polymer, such as Dimethicone, as an agent to improve the flow properties of HMG-CoA reductase inhibitors/Fluvastatin, Atorvastatin and/or to prevent sticking to parts of the processing machines, for example tableting machine and/or to protect and stabilize isomerization and/or oxidation susceptible pharmaceutical substances.

The following examples illustrate preferred embodiments in accordance with the present invention without limiting the scope or spirit of the invention:

### EXAMPLES

### Example 1

Tablet of 10 mg Atorvastatin

| Ingredients | Per Tablet |
|---|---|
| Atorvastatin | 10.85 mg |
| Dimethicone | 2.50 mg |
| Micr. Cellulose | 10.00 mg |
| Lactose monohydrate | 11.00 mg |
| Starch 1500 | 11.00 mg |
| Primojel | 4.00 mg |
| SLS | 0.50 mg |
| Aerosil | 0.50 mg |
| Mg stearate | 0.50 mg |
| Total weight: | 50.85 mg |

### Example 2

Tablet of 20 mg Atorvastatin

| Ingredients | Per Tablet |
|---|---|
| Atorvastatin | 21.69 mg |
| Dimethicone | 5.00 mg |
| Micr. Cellulose | 20.00 mg |
| Lactose monohydrate | 22.00 mg |
| Starch 1500 | 22.00 mg |
| Primojel | 8.00 mg |
| SLS | 1.00 mg |
| Aerosil | 1.00 mg |
| Mg stearate | 1.00 mg |
| Total weight: | 101.69 mg |

### Example 3

Tablet of 40 mg Atorvastatin

| Ingredients | Per Tablet |
|---|---|
| Atorvastatin | 43.38 mg |
| Dimethicone | 10.00 mg |
| Micr. Cellulose | 40.00 mg |
| Lactose monohydrate | 44.00 mg |
| Starch 1500 | 44.00 mg |
| Primojel | 16.00 mg |
| SLS | 2.00 mg |
| Aerosil | 2.00 mg |
| Mg stearate | 2.00 mg |
| Total weight: | 203.38 mg |

Tablets of the above formulations (Examples 1 to 3) were prepared according to the following manufacturing process: Atorvastatin and Dimethicone were admixed to complete homogeneity. The above mixture was passed through a sieve. The sieved mixture was then mixed with all the other excipients (Micr. Cellulose, Lactose monohydrate, Starch 1500, Primojel, SLS, Aerosil, Magnesium stearate) for about 15 minutes. The final mixture was then compressed directly into tablets in a tableting machine with round punches.

The bulk mixture showed satisfactory flow and could also be filled into capsules or sachets or compressed into tablets. The later solution was selected and the produced tablets were tested for hardness, friability, disintegration, and water content. All tests were performed according to European Pharmacopoeia 5.1 and were well within the specifications. Dissolution test in 900 ml water, 50 rpm Paddle Apparatus showed more than 70% dissolved in 10 min and more than 85% in 15 min.

### Example 4

Tablet of 80 mg Atorvastatin

| Ingredients | Per Tablet |
|---|---|
| Atorvastatin | 86.76 mg |
| Dimethicone | 20.00 mg |
| Micr. Cellulose | 80.00 mg |
| Lactose monohydrate | 88.00 mg |
| Starch 1500 | 88.00 mg |
| Primojel | 32.00 mg |
| SLS | 4.00 mg |
| Aerosil | 4.00 mg |
| Mg stearate | 4.00 mg |
| Total weight: | 404.76 mg |

Tablets of this formulation were prepared according to the following manufacturing process: Atorvastatin and ⅓ of the batch quantity of the Micr. Cellulose were added in Dimethicone and the formulation was admixed to complete homogeneity. The above mixture was passed through a sieve. The sieved mixture was then mixed with all the other excipients (⅔ of the batch quantity of the Micr. Cellulose, Lactose monohydrate, Starch 1500, Primojel, SLS, Aerosil, Magnesium stearate) for about 15 minutes. The final mixture was then compressed into tablets in a tableting machine with round punches.

### Example 5

Capsule of 20 mg Fluvastatin

| Ingredients | Per Capsule |
|---|---|
| Fluvasatin Sodium | 21.10 mg |
| Dimethicone | 5.00 mg |
| Micr. Cellulose | 18.00 mg |
| Lactose monohydrate | 22.45 mg |
| Starch 1500 | 22.45 mg |
| Primojel | 8.00 mg |
| SLS | 1.00 mg |
| Aerosil | 1.00 mg |
| Mg stearate | 1.00 mg |
| Total weight: | 100.00 mg |

### Example 6

Capsule of 40 mg Fluvastatin

| Ingredients | Per Capsule |
|---|---|
| Fluvastatin Sodium | 42.20 mg |
| Dimethicone | 10.00 mg |
| Micr. Cellulose | 36.00 mg |
| Lactose monohydrate | 44.90 mg |
| Starch 1500 | 44.90 mg |
| Primojel | 16.00 mg |
| SLS | 2.00 mg |
| Aerosil | 2.00 mg |
| Mg stearate | 2.00 mg |
| Total weight: | 200.00 mg |

Capsules of the formulations of the Examples 5 and 6 were prepared according to the following manufacturing process: Fluvastatin and Aerosil were accurately added in Dimethicone and the formulation was admixed to complete homogeneity. The above mixture was passed through a sieve. The sieved mixture was then mixed with all the other excipients (Micr. Cellulose, Starch 1500, Lactose monohydrate, Magnesium stearate, Primojel, SLS) and subsequently wet granulated using a water-free solvent specifically absolute ethanol, acetone or mixtures thereof. The wetted mass was then dried in 40° C., passed through a sieve to achieve the desired granule size and further mixed with Mg stearate.

Finally, it was formulated in a solid dosage form either by compressing it into a desired tablet form or by filling capsules and sachets.

The produced bulk mixture showed slightly better flow properties and could be formulated either as capsules or sachets or compressed into tablets. Capsules were produced and tested for content uniformity, disintegration, water content and dissolution proving that they are meeting the specifications.

These results demonstrate that dissolution profile remains unaffected besides the lower strength, proving that pharmacotechnical linearity i.e. proportional change in amount of excipients and active and total weight of the dosage form is achieved.

To provide a better homogeneity of the Fluvastatin and Atorvastatin in Dimethicone, small amounts of Aerosil is added to the formulation, which increases the suspendibility of the active ingredient in Dimethicone.

While the present invention has been described with respect to the particular embodiments, it will be apparent to those skilled in the art that various changes and modifications may be made in the invention without departing from the spirit and scope thereof, as defined in the appended claims.

1. A pharmaceutical composition for oral or sub-lingual administration comprising a HMG-CoA reductase inhibitor or a pharmaceutical acceptable salt thereof, as an active ingredient, and an effective amount of inorganic silica polymer such as Dimethicone as a stabilizer to inhibit isomerization and/or elimination and/or oxidation and/or re-crystallisation.

2. The pharmaceutical composition according to claim 1, wherein said inorganic silica polymer is Dimethicone.

3. The pharmaceutical composition according to claim 2, wherein it comprises from about 0.5 to 50% by weight of said HMG-CoA reductase inhibitor or salt thereof, and from about 0.1 to 25% by weight of said Dimethicone.

4. The pharmaceutical composition according to claim 2, wherein the weight ratio of said HMG-CoA reductase inhibitor or salt thereof to Dimethicone is preferably 500:1 to 1:50 and more preferably 200:7.5 to 7.5:80.

5. The pharmaceutical composition according to claim 1, wherein it comprises approximately 0.5% to 40%, more preferably 0.75% to 25% and most preferably 0.75% to 20% by weight of said HMG-CoA reductase inhibitor or salt thereof.

6. The pharmaceutical composition according to claim 2, wherein it comprises approximately 0.2% to 20%, more preferably 0.5% to 15% and most preferably 0.75% to 10% by weight of Dimethicone.

7. The pharmaceutical composition according to claim 1, wherein said HMG-CoA reductase inhibitor is Fluvastatin or a salt thereof. 8. The pharmaceutical composition according to any preceding claim, wherein said HMG-CoA reductase inhibitor is Atorvastatin or a salt thereof

9. The pharmaceutical composition according to claim 1, wherein it further comprises colloidal silicon dioxide

10. The pharmaceutical composition according to claim 1, wherein it further comprises at least one optionally excipient selected from the group consisting of diluents, binders, disintegrants, lubricants, and glidants.

11. The pharmaceutical composition according to claim 1, wherein said composition is in a solid dosage form such as a tablet, capsule or sachet comprising an active ingredient such as Fluvastatin or Atorvastatin or salts thereof.

12. A process for the preparation of a solid dosage form for oral or sub-lingual administration such as a tablet, capsule or sachet containing a HMG-CoA reductase inhibitor or a pharmaceutical acceptable salt thereof as an active ingredient and an effective amount of inorganic silica polymer such as Dimethicone as a stabilizer to inhibit isomerization and/or elimination and/or oxidation and/or re-crystallization, which comprises:
Forming a homogenous mixture by mixing the total quantity of said active ingredient with the total quantity of said Dimethicone;
Sieving the above mixture through a sieve;
Adding to the sieved mixture the total quantities of at least one optional excipient such as a binder, a diluent, a disintegrant, a lubricant and/or a glidant and mixing until uniform, and—Formulating the resulting mixture in a solid dosage form either by compressing it into a desired tablet form or by filling capsules or sachets.

13. A process for the preparation of a solid dosage form for oral or sub-lingual administration, such as a tablet, a capsule or a sachet, containing a HMG-CoA reductase inhibitor or a pharmaceutical acceptable salt thereof as an active ingredient and an effective amount of inorganic silica polymer such as Dimethicone as a stabilizer to inhibit isomerization and/or elimination and/or oxidation and/or re-crystallization, which comprises:
Forming a homogenous mixture by mixing the total quantity of said active ingredient and the total batch quantity or a portion thereof of an optional diluent, and/or the total batch quantity or a portion thereof of an optional filler;
Screening the above mixture through a screen and subsequently admixing with the total quantity of the effective amount of Dimethicone;
Sieving the above mixture on a sieve; —Adding to the sieved mixture the total quantity of any other optional excipient such as a binder, a disintegrant, a lubricant, a colorant and/or a glidant and mixing until uniform, and
Formulating the resulting mixture in a solid dosage form either by compressing it into a desired tablet form or by filling capsules or sachets.

14. A process for the preparation of a solid dosage form for oral or sub-lingual administration, such as a tablet, a capsule or a sachet, containing a HMG-CoA reductase inhibitor or a pharmaceutical acceptable salt thereof as an active ingredient and an effective amount of inorganic silica polymer such as Dimethicone as a stabilizer to inhibit isomerization and/or elimination and/or oxidation and/or re-crystallization, which comprises:
forming a first blend of the total quantity of said active ingredient with an effective amount of inorganic silica polymer, such as Dimethicone, as a stabilizer, and colloidal silicon dioxide and optionally the total batch quantity or a portion thereof of an optional diluent, and/or the total batch quantity or a portion thereof of an optional filler, and wet granulating by the addition of a water-free granulating medium such as absolute ethanol, acetone or mixtures thereof;
drying the wetted mass;
sieving the dried material to achieve the desired granule size
forming a second blend by mixing the total quantity of any other optional excipient such as a binder, a disintegrant, a lubricant, a colorant and/or a glidant until uniform and—adding and mixing the dried material with the second blend and
formulating the resulting mixture in a solid dosage form either by compressing it into a desired tablet form or by filling capsules or sachets.

15. The process according to claim 14, wherein said active ingredient is Fluvastatin or salt thereof.

16. The process according to claim 14, wherein said active ingredient is Atorvastatin or salt thereof.

17. A pharmaceutical composition—which is prepared by the process of claim 16.
